(19) United States
(12) Patent Application Publication
(10) Pub. No.: US 2005/0075595 A1
(43) Pub. Date: Apr. 7, 2005

(54) Adhesive dressings

(76) Inventor: Christopher William Hill, Nottingham (GB)

Correspondence Address: Gunnar G Leinberg;Nixon Peabody, Clinton Square, P O Box 31051, Rochester, NY, 14603-1051, US

(21) Appl. No.: 10/477,823

(22) PCT Filed: May 15, 2002
(86) PCT No.: PCT/GB02/02290

(30) Foreign Application Priority Data
May 15, 2001 (GB) 011828.0
Feb. 27, 2002 (GB) 0204570.6

(51) Int. Cl. A61F 13/00

(52) U.S. Cl. 602/41

(57) ABSTRACT

An adhesive film or membrane (1) is disclosed which is dismensioned for application to, and conformable to, the skin of a patient. The film or membrane (1) has an adhesive surface and carries, on that adhesive surface, a release liner (2) to mask the adhesive prior to use. The release liner (2) is formed with a generally central portion (6) which is releasable from the adhesive surface of the film or membrane (1) to permit application of dressing material (7) to the region of the adhesive surface so exposed, prior to complete removal of the release liner (2).

This invention relates to improvements in and relating to adhesive dressings, and in particular to adhesive components suitable for use in the preparation and application of so-called “island dressings” and to dressings incorporating such components.

Hospitals and medical practices have a need to stock dressings for the treatment of wounds in various conditions or stages of healing. The condition of a wound dictates the type of dressing to be applied, eg dry absorbent dressings, wet dressings (providing or maintaining moist conditions) and dressings incorporating anti-microbial additives.

Sometimes such dressings are secured in place using bandages. However, in many cases an adhesive fixation system is employed to secure the dressing in place. To facilitate the easy application of dressings, manufacturers provide dressings with an adhesive border and these are referred to as “island dressings”. Such dressings tend to be more expensive than dressings without such an adhesive border. Typically, they comprise a continuous film or membrane, to the central region of which the actual dressing material is affixed, and the peripheral region of which is coated with adhesive.

Hospitals and medical practices tend to carry a stock of numerous types of dressing. This is rather burdensome in terms of the need to keep adequate stocks of a wide range of products, and tends to increase the overall cost of healthcare provision.

It may also be desirable for the film or membrane material with the adhesive periphery to have varying properties. For instance, the material may be impermeable to air or moisture, providing an occlusive dressing, or it may be permeable. This further increases the range of products that it is necessary to stock.

There has now been devised improvements to adhesive dressings which overcome or substantially mitigate the above-mentioned and/or other disadvantages associated with the prior art.

According to a first aspect of the invention, there is provided an adhesive film or membrane, said film or membrane being dimensioned for application to, and conformable to, the skin of a patient, the film or membrane having an adhesive surface and carrying, on that adhesive surface, a release liner to mask the adhesive prior to use,
wherein the release liner is formed with a generally central portion which is releasable from the adhesive surface of said film or membrane to permit application of dressing material to the region of said adhesive surface so exposed, prior to complete removal of said release liner.

The adhesive film or membrane according to the invention is advantageous primarily in that it may be used in association with any one of a range of dressing materials to prepare a corresponding variety of dressings of the “island dressing” type. Further advantages and benefits of the invention, and in particular of preferred embodiments thereof, will become apparent from the following description.

The generally central portion of the release liner which is releasable from the adhesive surface of the film or membrane may have any suitable shape, but is most conveniently generally square or rectangular in form. In such a case, the central portion is preferably defined by cuts formed in at least three sides of the square or rectangle. Such cuts may be continuous cuts or may be interrupted by bridges to preserve the integrity of the release liner and prevent premature release of the central portion. The cuts may alternatively be of the nature of perforations. In general, all that is required is that the central portion should be easily peelable away from the film or membrane, but not so easily that it is liable to unintentional separation from the film or membrane.

The film or membrane may have any overall shape suitable for its intended application. For example, it may be circular or oval in shape. However, most commonly, the film or membrane will be generally straight-edged, ie generally square or rectangular in shape, though the corners are most preferably rounded.

The release liner is preferably formed of a generally conventional release paper. Such a release paper may comprise white paper coated on both sides with a polymer such as polyethylene for dimensional stability, or a glassine or clay coated paper. The side of each cover that is applied to the adhesive lower surface of the membrane film may be overcoated with a release coating, eg of silicone, to facilitate separation of the cover from the adhesive surface of the membrane or film.

In use, the central portion of the release liner is peeled away from the membrane film to expose an area of adhesive surface. The desired dressing material is then applied to the exposed adhesive surface. The central portion of the release liner, if not fully removed from the membrane or film, may be allowed to relax over the applied dressing material, providing a temporary protective shield for that material. Finally, the dressing is applied to the skin of the patient by removal of the release liner, thereby exposing the periphery of the adhesive surface of the membrane or film, around the dressing material, and pressing that adhesive periphery into engagement with the patient's skin.

The dressing material may be of any suitable form, including suitable materials that are conventionally used in such dressings. The material may, for instance, be an absorbent pad. The dimensions of the dressing material should be such that its size matches, or is less than, the area of the adhesive exposed by removal of the central portion of the release liner.

To facilitate application of the dressing to the patient's skin, the release liner is preferably formed in two parts, both parts preferably having portions that are free of the adhesive surface of the membrane or film such that those portions can be grasped and the respective part thereby removed.

The two parts of the release liner are preferably of differing sizes. Preferably, the first part of the release liner masks a minor part of the adhesive surface of the membrane or film. Most preferably, the first part is applied to a relatively narrow strip of the adhesive surface that extends across one edge of the assembly. By removal of the first part, a narrow part of the adhesive surface is exposed to enable the assembly to be securely positioned on the patient. The part of the adhesive surface exposed by removal of the first part is preferably a strip with a width of between 3 mm and 15 mm, eg about 5 mm.

The second part of the release liner preferably masks a major part of the adhesive surface of the membrane or film. The second part is preferably overlapped by the first part. The portion of the second part that is so overlapped is most preferably folded over such that when the first part is removed, the folded-over part of the second part is freed and serves as a tab by which the second part can be grasped. In such a case, the first part preferably overlies and extends a short distance beyond the folded over portion of the second part.

The membrane or film may be of sufficient thickness to be readily applicable to the skin of a patient without folding upon itself and without being generally difficult to manipulate. However, for many applications it is preferable for the membrane or film to be very thin. As such thin membranes lack independent rigidity, they tend to fold upon themselves. When the membrane folds upon itself in this way, the adhesive may cause the membrane to stick to itself. Due to the thin nature of the membrane, it may be difficult to peel the release liner away and apply the membrane to the wound site in a proper manner. For these reasons, the membrane or film may be supported by a carrier layer of greater rigidity.

The carrier layer is preferably adhered to the membrane or film by physical bonding, most preferably without the use of any adhesive or the like. By appropriate choice of materials for the carrier layer and the membrane or film a sufficient degree of adherence can be achieved such that the assembly maintains its integrity in use, yet the carrier layer can be relatively easily removed from the membrane or film after the membrane or film has been applied to the intended substrate (most commonly the skin of a patient).

Although the carrier layer may be described as relatively rigid, it will be appreciated that the rigidity of the carrier layer, though higher than that of the membrane or film, is nonetheless not necessarily high. The rigidity is generally sufficient to maintain the form of the assembly prior to and during application, ie to prevent the assembly curling or folding upon itself, but not so high as to inhibit the conformability of the assembly, ie the ability of the assembly to be applied to an irregularly shaped substrate.

The carrier layer preferably extends beyond the membrane or film by virtue of projecting at at least one of its edges beyond the membrane or film to form a tab by which the carrier layer can be grasped for removal from the membrane or film. Most preferably, the carrier layer projects in this way across the full extent of one of its edges. This is most preferably the edge parallel to that at which the minor part of the liner is applied. The width of the tab so formed is preferably sufficient to allow it to be easily grasped. Typically, the tab will have a width of between 3 mm and 15 mm, eg about 5 mm.

The carrier and the release liner, ie the combined first and second parts of the release liner where the release liner has such a construction, are preferably co-extensive, the release liner (most preferably, the second part thereof) preferably projecting beyond the membrane or film in a similar manner to the carrier.

In order to facilitate application of the adhesive film or membrane, and in particular to inhibit undesired removal of the adhesive film or membrane from the skin after application, when the carrier layer is removed, the adhesive film or membrane is preferably provided with a non-adhesive tab or the like by which it can be held in contact with the skin during removal of the carrier layer. Such a non-adhesive tab preferably extends across substantially the whole width of the adhesive film or membrane, most preferably at the edge at which the carrier layer projects beyond the membrane or film. The non-adhesive tab may, for instance, be a strip of material that overlaps with, and is attached to, the adhesive side of the film or membrane. By pressing the non-adhesive tab against the skin, while the carrier layer is peeled away, removal of the adhesive film or membrane from the skin is prevented.

It will generally be desirable for the non-adhesive tab to be removed after the carrier layer has been removed. To facilitate this, the film or membrane is preferably formed with a line of weakness adjacent to the non-adhesive tab so that the film or membrane may be easily broken at that line and the non-adhesive tab thereby removed. The line of weakness is most preferably a line of perforations extending across the width of the film or membrane.

Preferably, the membrane film and/or the carrier layer, and preferably both thereof, are transparent or substantially transparent, so that the substrate (eg the skin with a wound to which the membrane or film is to be applied) can be seen during application of the assembly to that substrate.

A particular advantage of a construction comprising both a thin membrane or film and a more rigid carrier is that the carrier may optionally be left in place, and not removed, in order to form an occlusive, non-permeable dressing. This further increases the versatility of the product.

The membrane or film (with its adhesive lower surface) may be moisture-permeable. The membrane or film may for instance have a moisture vapour transmission rate of at least 300 g/m²/24 h, more suitably at least 500 g/m²/24 h and preferably at least 700 g/m²/24 h at 37° C. at 100% to 10% relative humidity difference.

The membrane or film may contain apertures such as perforations to render it permeable to liquids such as water. However, whilst being permeable, the membrane or film is preferably substantially free of any apertures that might be sufficiently large to permit bacteria to penetrate through the membrane or film to the treatment site.

Materials used for the membrane or film can be any of a range of thin flexible films conventionally used as backings for wound dressings. Favoured flexible films are elastomeric moisture vapour permeable films. Favoured elastomeric moisture vapour permeable films include those formed from polyether-polyurethane and polyester-polyether copolymers.

The thickness of the membrane or film is preferably 9 to 80 μm, more suitably 15 to 50 μm, and preferably 20 to 40 μm, for example 30 μm.

The lower surface of the membrane or film is preferably rendered adhesive by having applied to it an adhesive coating. The adhesive is preferably any one of a range of readily available porous, pressure-sensitive adhesives which are non-irritant and not capable of inducing sensitisation in humans.

The adhesive coating can be any of a range of pressure-sensitive adhesives used on conventional wound dressings.

The adhesive coating can suitably have a thickness of 15 to 65 μm, and can preferably have a thickness of 20 to 40 μm. Such adhesive coatings will generally have a weight per unit area of 10 to 75 g/m², more usually of 15 to 65 g/m² and will preferably have a weight per unit area of 20 to 40 g/m².

The adhesive coating can be a continuous or a discontinuous coating, for example a patterned, porous or micro-porous coating.

The adhesive coating is, however, preferably continuous and moisture vapour permeable. Favoured adhesives are polyvinyl ether adhesives and acrylic adhesives.

The adhesive coating preferably has similar moisture-permeability to the membrane or film.

The carrier layer may be any of the flexible release materials used to protect the adhesive surface of a conventional adhesive dressing. Suitable carrier layers include plastics films such as polyethylene, polypropylene or unplasticised polyvinyl chloride, paper sheets and coated paper sheets which have been treated with a release agent such as silicone resin.

A particularly preferred material for the carrier layer is a polypropylene, particularly a bi-oriented polypropylene. It has been found that such material can be laminated to breathable polyurethane film with sufficient adherence to perform its function, yet can readily be released from the film after the film has been applied to the skin.

The thickness of the carrier layer is preferably 30 to 100 μm, more preferably 40 to 70 μm, eg 50 μm.

Currently preferred embodiments of the invention will now be described in greater detail, by way of illustration only, with reference to the accompanying drawings, in which

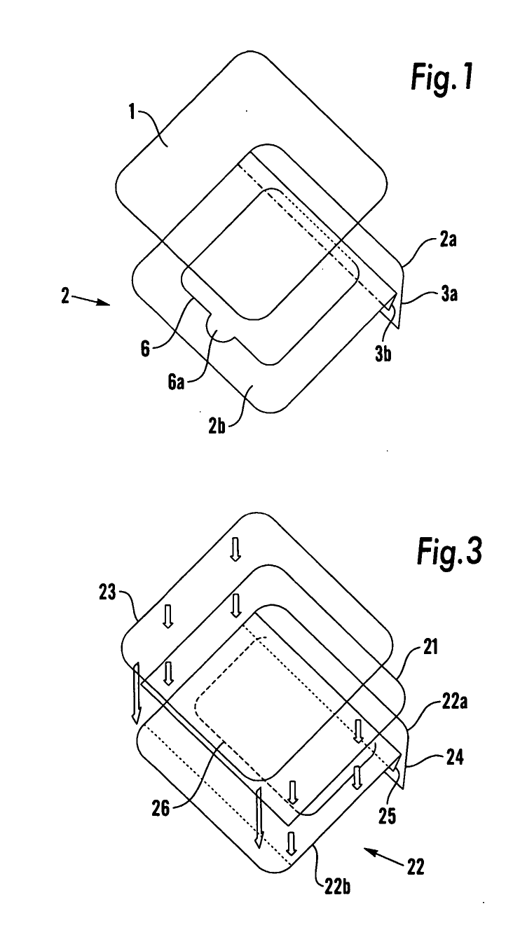

FIG. 1 is a perspective, exploded view of a first embodiment of an adhesive film according to the invention;

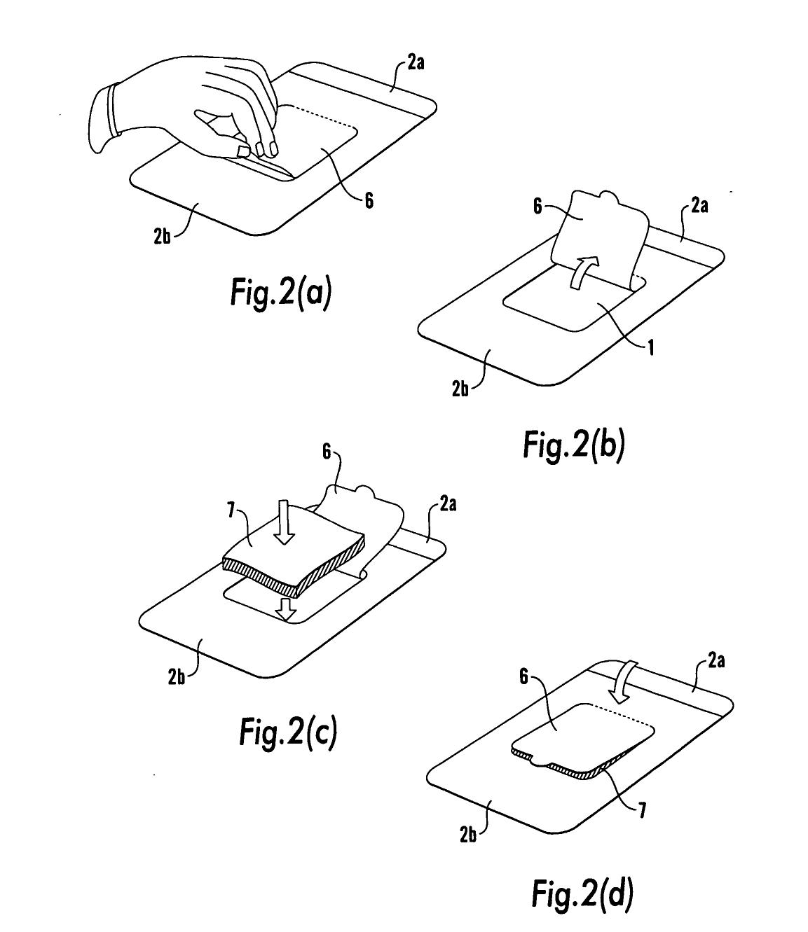

FIG. 2 shows steps in the use of the adhesive film of FIG. 1 in the preparation of an island dressing;

FIG. 3 is a view similar to FIG. 1 of a second embodiment of an adhesive film in accordance with the invention;

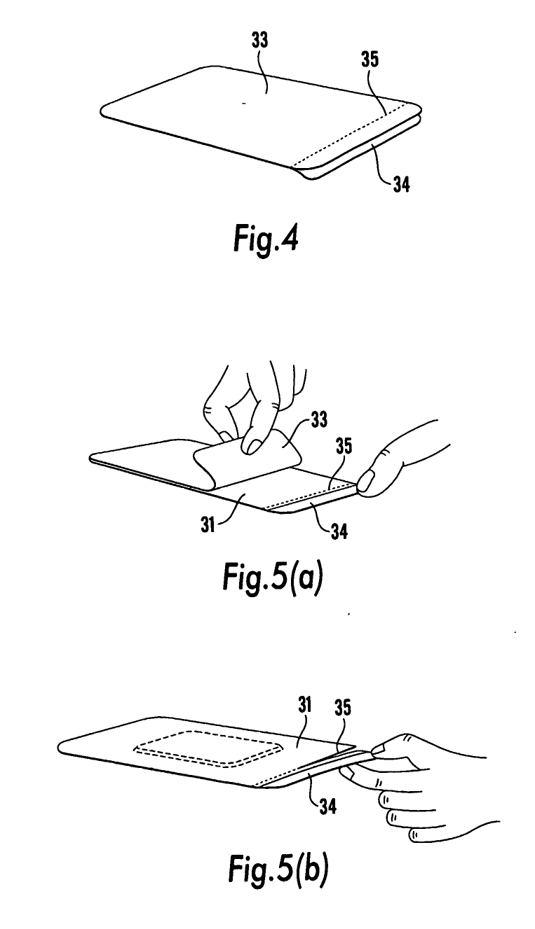

FIG. 4 is a perspective view of a modified form of the embodiment of FIG. 3; and

FIG. 5 shows final stages in the application of an island dressing utilising the embodiment of FIG. 4.

Referring first to FIG. 1, an adhesive membrane for use in the preparation of an island dressing comprises a polymeric film 1 with an adhesive lower surface and a two-part release liner 2 which is applied to the adhesive surface so as to mask the adhesive prior to use.

The film 1 is generally square, with rounded corners, and comprises a synthetic polymeric material such as polyethylene, polypropylene or polyvinylchloride. The film has a thickness of around 50 μm.

The undersurface (as viewed in FIG. 1) of the film 1 is rendered adhesive by being coated with a layer of pressure-sensitive adhesive, eg a layer of 25 g/m² acrylate adhesive.

The adhesive layer is masked by minor and major parts (2a,2b respectively) of the release liners 2. The minor part 2a masks a 5 mm wide strip of adhesive along one edge of the film 1. The major part 2b masks the remainder of the adhesive. The two parts 2a,2b of the liner 2 are together co-extensive with the film 1.

The minor part 2a of the liner 2 overlaps the major part 2b, the portion of the latter which is overlaid by the minor part 2a being folded over to form a tab 3b, and similarly the portion of the minor part 2a which overlaps the major part 2b forming a tab 3a.

A generally square (but with rounded corners) flap 6 is formed in the major part 2b of the liner 2, defined by cuts along three sides, the uncut side being that adjacent to the junction of the major and minor parts 2a,2b of the liner 2. The edge of the flap 6 opposite to the uncut edge is formed with a projecting, generally semicircular tab 6a.

The manner in which the adhesive membrane is used to prepare an island dressing will now be described with reference to FIG. 2. First, the tab 6a is lifted (FIG. 2a) and used to peel back the flap 6 (FIG. 2b). A pad or the like of dressing material 7, eg absorbent material, is then applied to the generally central area of the adhesive surface of the film 1 that is so exposed (see FIG. 2c). The flap 6 is then allowed to relax and temporarily shields the dressing material 7, as shown in FIG. 2d. This provides some temporary protection against contamination of the dressing material 7.

The dressing assembly so formed can then be applied to the skin of a patient, eg to cover a wound, by first grasping the tab 3a and peeling off the minor part 2a of the liner 2. This exposes a narrow strip of adhesive along one edge of the film 1. This exposed strip is applied to the patient's skin in the desired position adjacent the wound. The tab 3b is then grasped and used to peel off the major part 2b of the liner 2, including the flap 6. The film 1 is then pressed down into engagement with the patient's skin, the periphery of the adhesive surface adhering to the patient's skin and holding the dressing material in place.

A second embodiment of an adhesive membrane according to the invention is shown in FIG. 3. This differs from the embodiment of FIG. 1 in that it comprises, in addition to a thin film 21 and a two-part liner 22, also a carrier layer 23. The assembly thus comprises three layers. The topmost layer (as viewed in FIG. 3) is the carrier layer 23, which has dimensions of 100×130 mm and thickness 50 μm. The carrier layer 23 is of bioriented polypropylene. The carrier layer 23 is bonded to the film 21, which is 30 μm thick and of polyurethane. The film 21 is 5 mm shorter than the carrier layer 23 so that the carrier layer 23 projects beyond one edge of the film 21 by that distance.

The underside of the film 21 is coated with a layer of 25 g/m² acrylate adhesive by which, in use, the film 21 is adhered to a substrate, which is most commonly the skin of a patient.

The adhesive layer is masked by minor and major release liners 22a,22b. The minor release liner 22a masks a 5 mm wide strip of adhesive along the edge of the assembly that is opposite to the projecting part of the carrier layer 23. The major liner 22b masks the remainder of the adhesive. The two parts of the liner 22a,22b are together co-extensive with the carrier layer 23, the major part 22b projecting beyond the film 21 in a similar manner to the carrier layer 23.

The minor liner 22a overlaps the major liner 22b, the part of the latter which is overlaid by the minor liner 22a being folded over to form a tab 25 and similarly the part of the minor liner 22b which overlaps the major liner 22b forming a tab 24.

As for the first embodiment described above, a flap 26 is formed in the generally central area of the major liner 22b, by means of cuts defining three sides of the generally square flap 26.

The second embodiment is used in a similar manner to the first, save that following removal of the liner 22 and securing of the periphery of the film 21 to the patient's skin, the carrier layer 23 can be removed, by grasping the projecting edge of the carrier layer 23 and peeling it away from the film 21.

The second embodiment is beneficial in that it offers the advantages of very thin-film membrane dressings, the carrier layer 23 however facilitating manipulation and application of the dressing. It will, however, be appreciated that the carrier layer 23 could be left in place, eg if an occlusive dressing is required.

An improvement of the second embodiment is shown in FIGS. 4 and 5. Like the second embodiment, this embodiment comprises a thin film 31, a two-part liner (not visible in FIGS. 4 and 5) and a carrier layer 33. This embodiment differs from the second embodiment, however, in that a tab 34 of non-adhesive material (actually a strip of material similar to that used for the liner) is connected to the thin film 31 at the edge of the thin film 31 beyond which the carrier layer 33 projects. The tab 34 underlaps the adhesive surface of the thin film 31 and is hence bonded to the thin film 31.

The third embodiment is used in a similar manner to the second. A flap in the major liner is peeled back and a pad of dressing material is applied to the adhesive surface so exposed. The minor liner is removed and the dressing assembly positioned on the patient's skin. The major liner is then removed and the assembly pressed down onto the skin.

If it is then desired to remove the carrier layer 33, this is carried out by grasping the part of the carrier layer that projects beyond the thin film 31 and peeling the carrier layer 33 off. To counteract any tendency for the adhesion between the carrier layer 33 and the thin film 31 to pull the thin film 31 away from the skin, the tab 34 is held down while the carrier layer 33 is removed, as shown in FIG. 5a.

Once the carrier layer 33 has been removed, the tab 34 can be removed. This is facilitated by a line of perforations 35 that is punched through the whole assembly close to the junction of the tab 34 and the thin film 31. Simply by grasping the tab 34 and pulling it as indicated in FIG. 5b the tab 34 and the terminal part of the thin film 31 to which the tab 34 is attached can be broken away along the perforations 35 and removed. It will be appreciated that where the carrier layer 33 is not removed, so as to form an occlusive dressing, then the tab 34 and the part of the carrier layer 33 that projects beyond the thin film 31 may similarly be removed by breaking along the line of perforations 35.

It will also be appreciated that all the above embodiments, while suitable for use in the assembly of an island dressing, are not restricted to that application. The second embodiment, for instance, may be used as a conventional thin film dressing. In such a case, the minor liner 22a is removed and the exposed adhesive strip applied to the patient's skin. The major liner 22b, including the flap 26, is then removed and the membrane pressed down in the usual way. The carrier layer 23 can then be removed (or left in place, as desired).

1. A method for the preparation of a dressing for application to the skin of a patient, which method comprises:
providing an adhesive film or membrane, said film or membrane being dimensioned for application to, and conformable to, the skin of the patient, the film or membrane having an adhesive surface and carrying, on that adhesive surface, a release liner to mask the adhesive prior to use,
wherein the release liner is formed with a generally central portion which is releasable from the adhesive surface of said film or membrane;
releasing said generally central portion of the release liner from the adhesive surface of said film or membrane to expose a region of said adhesive surface;
applying dressing material to the region of said adhesive surface so exposed; and
completely removing said release liner.

2. A method according to claim 1, wherein the central portion of the release liner is generally square or rectangular in form.

3. A method according to claim 2, wherein the central portion is defined by cuts formed in at least three sides of the square or rectangle.

4. A method according to claim 3, wherein the cuts are perforated or interrupted by bridges to preserve the integrity of the release liner and prevent premature release of the central portion.

5. A method according to claim 1, wherein the film or membrane is substantially straight-edged.

6. A method according to claim 5, wherein the corners of the film or membrane are rounded.

7. A method according to claim 1, wherein the release liner is formed of paper.

8. A method according to claim 7, wherein the release liner comprises white paper coated on both sides with a polymer for dimensional stability.

9. A method according to claim 7, wherein the release liner comprises a glassine or clay coated paper.

10. A method according to claim 1, wherein the side of the release liner that is applied to the adhesive surface of the film or membrane film is overcoated with a release coating to facilitate separation of the liner from the adhesive surface of the membrane of film.

11. A method according to claim 10, wherein the release coating is silicon.

12. A method according to claim 1, wherein the release liner is formed in two parts, both parts having portions that are free of the adhesive surface of the membrane or film such that those portions can be grasped and the respective part thereby removed.

13. A method according to claim 12, wherein the two parts of the release liner are of differing sizes, the first part of the release liner masking a minor part of the adhesive surface of the membrane or film and the second part of the release liner masking a major part of the adhesive surface of the membrane or film.

14. A method according to claim 13, wherein the first part is applied to a strip of the adhesive surface that extends across one edge of the assembly.

15. A method according to claim 14, wherein the part of the adhesive surface exposed by removal of the first part has a width of between 3 mm and 15 mm.

16. A method according to claim 13, wherein the second part is overlapped by the first part.

17. A method according claim 16, wherein the portion of the second part that is overlapped by the first part is folded over such that when the first part is removed, the folded-over part of the second part is freed and serves as a tab by which the second part can be grasped.

18. A method according claim 17, wherein the first part overlies and extends a short distance beyond the folded over portion of the second part.

19. A method according to claim 1, wherein the membrane or film is supported by a carrier layer of greater rigidity.

20. A method according to claim 19, wherein the carrier layer is adhered to the membrane or film by physical bonding.

21. A method according to claim 19, wherein the carrier layer extends beyond the membrane or film at least one of its edges to form a tab by which the carrier layer can be grasped for removal from the membrane or film.

22. A method according to claim 21, wherein the carrier layer projects across the full extent of one of its edges.

23. A method according to claim 21, wherein the tab has a width of between 3 mm and 15 mm.

24. A method according to claim 19, wherein the carrier layer and the release liner are co-extensive.

25. A method according to claim 24, wherein the release liner projects beyond the membrane or film.

26. A method according to claim 21, wherein the adhesive film or membrane is provided with a non-adhesive tab by which it can be held in contact with the skin during removal of the carrier layer, the non-adhesive tab extending across substantially the whole width of the adhesive film or membrane at the edge at which the carrier layer projects beyond the membrane or film.

27. A method according to claim 26, wherein the non-adhesive tab is a strip of material that overlaps with, and is attached to, the adhesive side of the film or membrane.

28. A method according to claim 27, wherein the film or membrane is formed with a line of weakness adjacent to the non-adhesive tab so that the film or membrane may be easily broken at that line and the non-adhesive tab thereby removed.

29. A method according to claim 19, wherein the membrane film and the carrier layer are transparent or substantially transparent.

30. A method according to claim 1, wherein the membrane or film with its adhesive lower surface is moisture-permeable.

31. A method according to claim 30, wherein the membrane or film has a moisture vapor transmission rate of at least 300/m²/24 h at 37° C. at 100% to 10% relative humidity difference.

32. A method according to claim 31, wherein the membrane or film has a moisture vapor transmission rate of at least 700g/m²/24 h at 37° C. at 100% to 10% relative humidity difference.

33. A method according to claim 30, wherein the membrane of film comprises apertures to render it permeable to liquids such as water.

34. A method according to claim 30, wherein the membrane or film is formed from polyether-polyurethane or polyester-polyether copolymers.

35. A method according to claim 1, wherein the thickness of the membrane of film is in the range 9 to 80 um.

36. A method according to claim 35, wherein the thickness of the membrane or film is in the range 20 to 40 um.

37. A method according to claim 1, wherein the lower surface of the membrane or film is rendered adhesive by having applied to it an adhesive coating.

38. A method according to claim 37, wherein the adhesive coating has a thickness in the range 15 to 65 um.

39. A method according to claim 38, wherein the adhesive coating has a thickness in the range 20 to 40 um.

40. A method according to claim 37, wherein the adhesive coating has a weight per unit area in the range 10 to 75 g/m².

41. A method according to claim 40, wherein the adhesive coating has a weight per unit area in the range 20 to 40 g/m².

42. A method according to claim 37, wherein the adhesive coating is of a polyvinyl ether adhesive or acrylic adhesive.

43. A method according to claim 19, wherein the carrier layer is a film of polyethylene, polypropylene or unplasticised polyvinyl chloride.

44. A method according to claim 19, wherein the carrier layer is a paper sheet.

45. A method according to claim 43, wherein the carrier layer is of polypropylene.

46. A method according to claim 45, wherein the carrier layer is of bi-oriented polypropylene.

47. A method according to claim 19, wherein the carrier layer has a thickness in the range 30 to 100 um.

48. A method according to claim 47, wherein the carrier layer has a thickness in the range 40 to 70 um.
